(19) United States
(12) Patent Application Publication
(10) Pub. No.: US 2003/0092995 A1
(43) Pub. Date: May 15, 2003

(54) System and method of positioning implantable medical devices

(75) Inventor: David L. Thompson, Andover, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/087,949

(22) Filed: Feb. 28, 2002

Related U.S. Application Data
(60) Provisional application No. 60/338,288, filed on Nov. 13, 2001.

(51) Int. Cl. A61B 5/05; A61B 6/00

(52) U.S. Cl. 600/473

(57) ABSTRACT

A device and method for deploying a lead in tortuous pathways such as the left ventricle or the coronary sinus vasculature. A catheter having fiber optics is inserted into the vasculature of a patient with the aid of a guide wire or equivalent. The fiber optics transmits infrared light to an optical head at the distal end of the catheter. Light reflecting from the vasculature system is collected and transmitted through the optical fibers to an infrared camera for imaging. Alternatively, an active pixel sensor is positioned distally on the lead for collecting and transmitting the image to a display device located external to the patient. Proper selection of the wavelength of infrared illumination allows objects to be imaged through bodily opaque fluid. Another embodiment enables viewing of a cardiac lead for extraction by a laser. Yet another embodiment enables proper positioning of an ablation catheter before energization.

This application claims priority to U.S. Provisional Patent Application No. 60/338,288 filed Nov. 8, 2001, and incorporates the specification and drawings in their entireties by reference herein.

### FIELD OF THE INVENTION

This invention relates to implantable medical device systems, and in particular to a device and method for the placement and control of an implantable medical device into specific cardiovasculature locations such as the distal vasculature system of the coronary sinus.

### BACKGROUND

The use of implantable medical electrical stimulation and/or sensing leads is well known in the fields of cardiac stimulation and monitoring. Endocardial leads are placed through a transvenous route to place one or more sensing and/or stimulation electrodes in a desired location within a heart chamber or interconnecting vasculature. In order to achieve reliable sensing of the cardiac electrogram and/or to apply stimulation that effectively paces, cardioverts, or defibrillates a cardiovascular structure, it is necessary to accurately position the electrode surface against the endocardium, pericardium, or within the myocardium, or at a desired location within the venous system. This precise positioning may be accomplished using a delivery system that may include a guide catheter, stylet, guidewire, steerable sheath, and/or an equivalent delivery mechanism.

It has long been known that leads and/or other implantable medical devices (IMD) may be positioned within the right atrium and/or ventricle to provide therapies for cardiac ailments. Recently, it has become more apparent that certain cardiac disfunctions such as heart failure may be effectively treated by positioning leads, catheters, and/or IMDs adjacent to, or within, the left side of the heart. For example, cardiac resynchronization therapy may be accomplished by pacing both the left and right ventricles. Left ventricular pacing pulses may be delivered via a lead positioned within the coronary sinus or a branch cardiac vein in proximity to the left ventricle. To position a lead or catheter so this type of treatment may be provided, a distal end of the device is advanced through the superior vena cava, into the right atrium, through the valve of the coronary sinus, and into the coronary sinus. The device may be further advanced into a coronary vein communicating with the coronary sinus, such as the cardiac great vein, the middle cardiac vein, the posterial lateral cardiac vein, or the anterial lateral cardiac vein as examples. Additionally, it may be desirable to locate pacing leads, or other types of leads, at other locations within the heart for various reasons. For example, it may be desirable to locate such leads in the right ventricular outflow tract (RVOT), the Bundle of His, or the triangle of Koch, as such alternates may enhance the effectiveness of the heart therapy delivered to such sites.

Several common delivery systems have been developed to place electrodes in a specific location, such as, within the left side of the heart. According to one approach, a guide catheter is navigated into the desired location in the vasculature. A lead is then fed through the inner lumen of the catheter such that the lead electrode(s) are positioned at predetermined locations. The guide catheter may then be withdrawn. This type of approach is described in commonly assigned U.S. Pat. No. 6,006,137, 5,246,014, and 5,851,226 incorporated herein by reference in their entireties.

Another approach to lead placement involves the use of a guide wire that is steered into a desired location within the vasculature. The lead body is then tracked over the wire and the wire is withdrawn. According to this design, the guide wire passes through an inner lumen of the lead for at least a portion of a length of the lead. A similar approach is described in commonly assigned U.S. Pat. No. 5,902,331 to Bonner et al., also incorporated herein by reference in its entirety. The disclosed system includes a pusher mechanism that is adapted to slidably engage a guidewire that has previously been placed at a desired implant site. The pusher mechanism couples to a lead body to allow the pusher to guide the lead over the guidewire to the desired implant site.

Regardless of which of the above-described delivery systems is utilized, a significant challenge involves the location and navigation of a guide device, such as a catheter or guide wire, into the coronary sinus. Anomalies in the vascular anatomy, their small size, and the number of branch veins associated with the anatomy make locating the desired path challenging.

One mechanism used to aid in placement of a device within the coronary sinus involves the use of radiopaque dye. This dye may be injected into the venous anatomy so that the chambers of the heart and the related vasculature system are visible using a fluoroscopic device. This procedure, sometimes referred to as a “venogram”, allows the surgeon to locate the coronary sinus, its distal vasculature, or other anatomical structure when performing an implant procedure.

It may be undesirable to use fluoro visible media during an implant process for several reasons. First, some patients experience adverse physical reactions when exposed to the fluoro visible dye used to obtain a venogram. In these situations, an alternative approach is needed to accomplish lead placement. Moreover, the use of fluoroscopy exposes the patient, implant surgeon and assistants to radiation. The use of protective lead aprons prevents or limits exposure to the physician and his attending staff, but their heavy weight is problematic for long and/or several procedures daily. Additionally, a fluoroscope of the type needed for obtaining the fluoro-visible image may not be available. Finally, obtaining the venogram adds additional steps to the implant procedure, lengthening the time required to complete the procedure, increases the cost of the procedure and increases the risk of infection and complications to the patient.

Another approach to performing catheter placement and cardiac surgery is disclosed in U.S. Pat. No. 6,178,346 incorporated herein by reference in its entirety. That patent describes the use of an infrared imagining system that is capable of transmitting light into an environment containing opaque or semi-opaque fluids such as blood. However, a lead delivery method is not disclosed.

What is needed, therefore, is an alternative system and method for accurately and rapidly placing implantable medical leads and sensors at precise locations within the vascular system of the body such as within the coronary sinus or a branch vein without the need to inject a fluoro visible media into the body and to minimize the use of fluoroscopy.

### SUMMARY OF THE INVENTION

The present invention provides a visual catheter guide system to navigate and position a medical device within the coronary sinus and branch veins, such as the cardiac great vein, the middle cardiac vein, the posterial lateral cardiac vein, the anterial lateral cardiac vein and similar other cardiac vasculature. The system enables continuous visual imaging of the deployment, location and vascular environment of the catheter inside the cardiac network of vasculatory system.

In one aspect of the invention, a shaft adapted to be positioned within the cardiac vein or coronary artery incorporates a fiber optic cable suitable for transmitting light. Preferably, an infrared light source transfers infrared light down the cable. An optical head assembly coupled to the cable is implemented as a transceiver for the infrared light. Further, sensing systems receive the infrared light from the body using optical assemblies. An image is generated indicating the position of the distal portion of the elongated shaft, in addition to monitoring navigation on a real-time visual basis.

Various systems of lead placement in the coronary sinus are advanced in the present invention. The vision system, for example, may incorporate an ablation system for navigation and placement of an ablation electrode in a human heart. Another embodiment includes an implementation of a laser lead extraction system to view and remove a cardiac lead from the heart. Further, proper positioning of an ablation catheter prior to the application of ablation energy is enabled using the scheme and structure of the present invention.

### BRIEF DESCRIPTION OF THE DRAWINGS

These and other features of the present invention will be more readily understood from the following detailed description of the preferred embodiments thereof, when considered in conjunction with the drawings, in which like reference numerals indicate identical structures throughout the several views, and wherein:

FIG. 1 depicts an infrared endoscope system connected to a catheter delivery system having a distal end positioned in a coronary vein;

FIG. 2 depicts a catheter delivery system of the present invention;

FIG. 3A is a cross section of the catheter delivery system of FIG. 1;

FIG. 3B is a cross section of a first alternative embodiment of the catheter delivery system of FIG. 1;

FIG. 3C is a cross section of a second alternative embodiment of the catheter delivery system of FIG. 1;

FIG. 4 is a cross section of an alternative implementation of the catheter delivery system of FIG. 1;

FIG. 5 is an alternative delivery system for use with the vision system of FIG. 1; and

FIG. 6 depicts an additional alternative embodiment of the present invention.

### DETAILED DESCRIPTION OF SPECIFIC EMBODIMENTS

As described herein above in the background, the delivery and positioning of this left ventricular lead in the coronary sinus vasculature system is problematic. The claimed invention allows the rapid, accurate and easy deployment and positioning of a lead in the coronary sinus, distal coronary sinus, great cardiac vein, the middle cardiac vein, the posterial lateral cardiac vein, the anterial lateral cardiac vein or other distal vasculature.

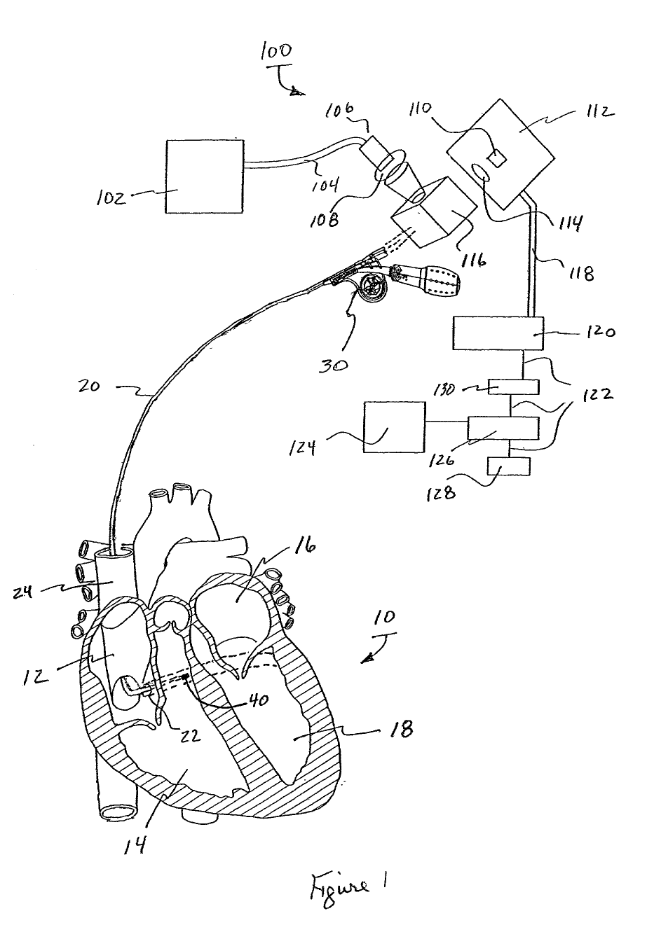

FIG. 1 illustrates a human heart 10 in cross section with the right atrium 12, right ventricle 14, left atrium 16, left ventricle 18, coronary sinus ostium 22 and superior vena cava 24 shown.

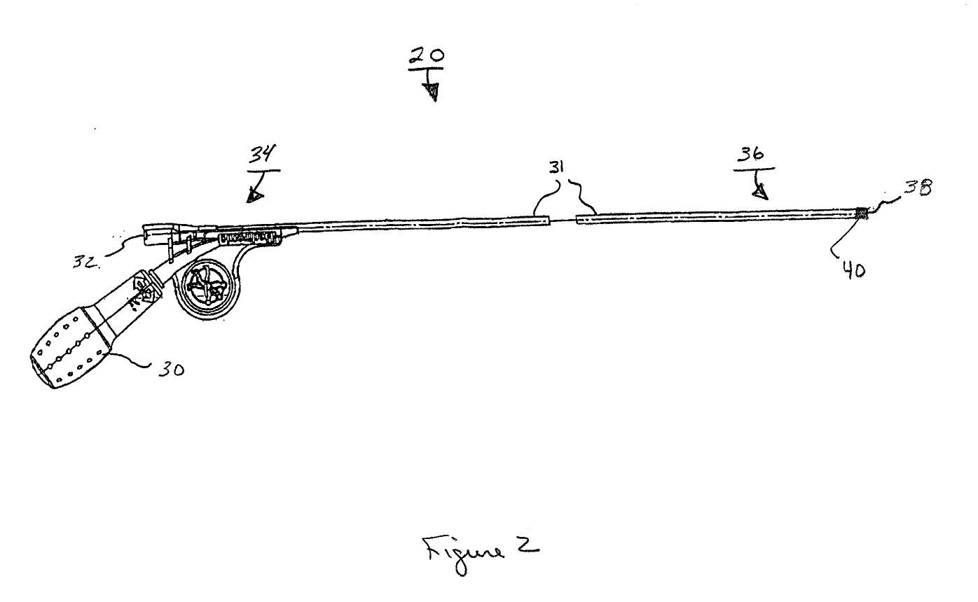

FIG. 1 further illustrates the catheter guide system 20 shown in FIG. 2 disposed within the patient's vascular system with the distal section (not shown) of the catheter seated within the patient's coronary sinus ostium 22. In this embodiment, the catheter guide system 20 has been introduced from the cephalic vein (not shown) and advanced through the superior vena cava 24 and into the right atrium 12.

FIG. 1 still further shows catheter guide system 20 coupled to a system 100 for transferring infrared light to the distal end of the catheter, capturing the reflected light, and displaying an image for use by an implanting physician. This catheter uses technology disclosed in U.S. Pat. No. 6,178,346, incorporated herein by reference, to transmit light through opaic fluids such as blood. System 100 includes a laser diode 106 and infrared camera 112. Light reflected from within the coronary sinus is received by an optical head 40 (located distally on catheter 20) and is transmitted via optical fibers in lumens contained in catheter 20 to beamsplitter 116. Thereafter, the light is passed through camera optics 114 to a sensing device such as infrared camera 112. There, the light is detected by the infrared camera sensor 110 and converted to an electronic signal. This signal is relayed via camera cable 118 to an image-processing unit 120. This unit uses known image processing techniques to enhance the image created by the reflected and scattering light to provide a view of the cardiac vasculature. The image processing unit 120 is connected with electrical cable 122 to the central processing unit or CPU 130, which reconfigures the signals and transmits these signals through an electrical cable 122 to a video processor 126 which processes the signals for video imaging. A video console 124 and video recorder 128 may also be coupled to the video processor 126.

The system of FIG. 1 may be used to position catheter guide system 20 within the coronary sinus and branch veins such as the cardiac great vein, the middle cardiac vein, the posterial lateral cardiac vein, or the anterial lateral cardiac vein. Using a light having wavelengths starting in the 1.5-1.8 micron range, structures may be visualized at a distance of 4-5 millimeters through blood. Wavelengths of approximately 2.1 microns would also be suitable for this embodiment. This region permits viewing arterial structures about 10 millimeters through blood. Higher wavelength regions (e.g., 3.8-4.4, 4.7-5.3, and 7-10) would generate more accurate images but also result in more rigid catheter designs because of the larger-sized optical fibers required in this embodiment.

As shown in FIG. 2, in one embodiment, the invention includes a guide catheter system 20 such as disclosed in U.S. Pat. No. 6,021,340, incorporated herein by reference in its entirety. The catheter includes an elongated shaft 31, a distal shaft section 36, a proximal shaft section 34, an inner lumen 32 and a control handle 30 on the proximal end of the shaft 31. A port 38 is provided in the distal end of the catheter shaft 36 that is in fluid communication with the inner lumen 32. The distal shaft section 36 is controllable in a 3D manner via the proximal handle 30 as described in the '340 patent. The catheter shaft 31 contains optical fibers to transmit infrared light from the vision system 100 to a lens 40 and transmits reflected light back to the vision system 100 for processing and displaying vasculature structures. Catheter 20 may optionally contain distally located sense/pace electrodes for the verification and confirmation of proper location.

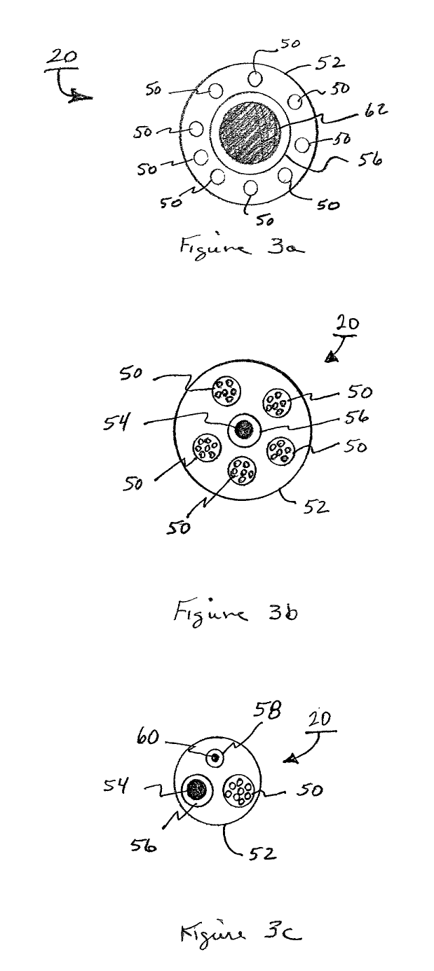

FIG. 3A shows a cross sectional view of the guide catheter system 20 of FIGS. 1 and 2. The catheter body 52 contains several lumens 50 containing optical fibers for the transmission of infrared light from a proximal source 106 to a lens 40 on the distal end of the catheter and for the reflected light to be returned to the infrared camera 112. A pacing or defibrillation lead 62 is shown in a central lumen 56. The catheter guide system 20 is used to deliver the distal end of the catheter to a desired location and then a lead is threaded through lumen 56 to deliver the lead to the proper location. This method allows the use of very small diameter lead systems because a stylet lumen is not required in the lead body construction.

FIG. 3B shows an alternative cross sectional view of the guide catheter system 20 of FIGS. 1 and 2. The catheter body 52 contains several lumens 50 containing optical fibers for the transmission of infrared light from a proximal source 106 to the distal end of the catheter and for the reflected light to be returned to the infrared camera 112. A guide wire 54 is shown in a central lumen 56. This method allows the distal end of a guide wire to be positioned in the correct location, a lead body is tracked over the wire and the wire is then withdrawn.

FIG. 3C shows a cross sectional view of the guide catheter system 20 of FIGS. 1 and 2 containing an alternative embodiment of catheter body construction. The catheter body 52 contains a single lumen 50 containing optical fibers for the transmission of infrared light from a proximal source 106 to the distal end of the catheter. At the distal end of the catheter 38 an active pixel sensor is positioned to receive light reflected from the coronary sinus vasculature system. The active pixel sensor is as substantially described in U.S. Pat. Nos. 6,204,524, 6,243,131, and published application No. 2001/0055832. The '524 and '131 patents and '832 application are incorporated herein by reference in their entireties. Lumen 58 contains several insulated electrical wires 60 for providing power to the sensor and return signals depicting the field of view of the active pixel sensor. A guide wire 54 is shown in a central lumen 56. The operation of delivering a lead via a properly positioned guide wire is as described above. This catheter design allows for a smaller, more flexible catheter design allowing it to reach smaller and more distal vasculature. Additionally, larger optical fibers may be used to allow a larger wavelength infrared light source (i.e., 3.8 4.4, 4.7-5.3, and 7-10 microns) to be used providing for images with improved clarity and increased accuracy.

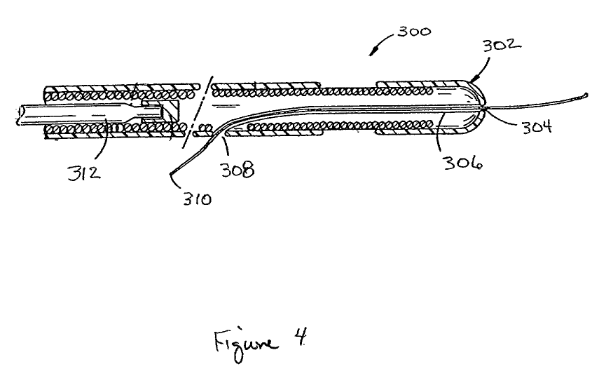

FIG. 4 depicts an alternative method of lead placement in the coronary sinus (see FIGS. 1, 2, 3B and 3C). In this method, a guide wire is advanced into the coronary sinus ostium and advanced through the vasculature system to the proper location for pacing and defibrillation lead placement as is well known in the art. The guide wire 314 is advanced under visual control by the system shown in FIG. 2 and herein described above. After guide wire placement, an over the wire pacing or defibrillation lead 306 can be inserted and then the lead passed over the guide wire 314 through lumen 322 until it is properly positioned. The guide wire 314 enters the distal end of the lead 306 through lumen 322 at the lead distal tip 324 and exits the side of the lead 306 via exit port 316. After the lead is properly positioned, the guide wire 314 can then be retracted from the lead 306.

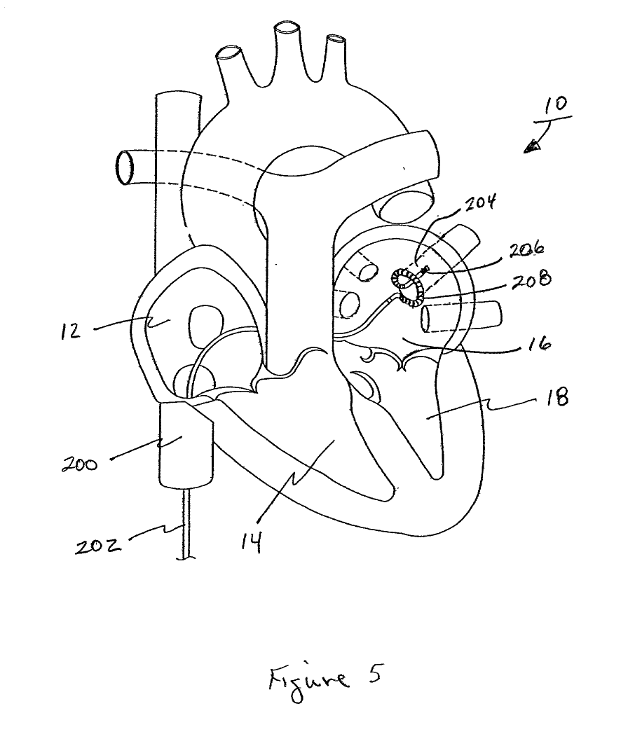

FIG. 5 depicts another embodiment, whereby the vision system described above is incorporated into an ablation system as described in U.S. Pat. No. 6,325,797 for accurate location of the ablation electrode in a human heart 10. The '797 patent describes a catheter assembly and method for treatment of cardiac arrhythmia, for example, atrial fibrillation, by electrically isolating a vessel, such as a pulmonary vein 204, from a chamber, such as the left atrium 16. The catheter assembly includes a catheter body 202 and at least one electrode 208. The distal portion of the catheter 202 extends from an intermediate portion (inserted from the inferior vena cava 200) and forms a substantially closed loop transverse to the longitudinal axis with the at least one electrode 208 disposed along the loop. With this configuration, the loop is axially directed into contact with the chamber wall about the pulmonary vessel ostium 204. Upon energization, the electrode ablates a continuous lesion pattern about the vessel ostium 204, thereby electrically isolating the vessel from the chamber. In the herein described embodiment, additional lumens filled with optical fibers may be employed as described above to allow the visualization of the location of the catheter 202 via a lens 206 located at the distal end of catheter 202. The visualization will allow the proper positioning of the ablation electrode(s) in a fixed and proper location (for example, the pulmonary vein ostium 204).

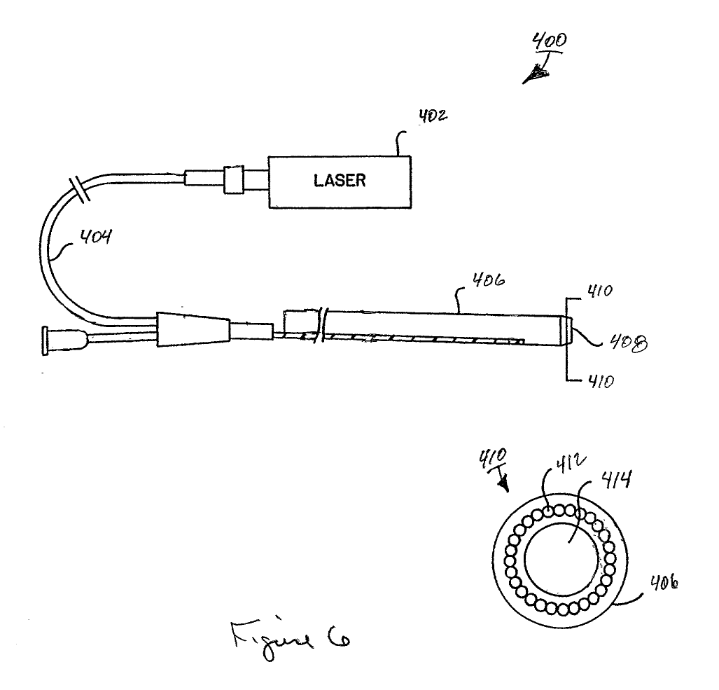

FIG. 6 depicts yet another embodiment incorporating the vision system as described above into a lead extraction device as substantially described in U.S. Pat. Nos. 5,423,806 and 5,674,217, both to Wahlstrom, et al. which utilizes laser light to separate an implanted object, such as a pacemaker lead, from fibrous scar tissue and thereby permit the implanted object to be extracted from a body. The extraction device features a catheter 406 having a central lumen 414 dimensioned so a pacemaker lead will fit within. The catheter 406 is thereby guided by the lead. The catheter 406 has at least one optical fiber 412 to emit laser light 402 from the distal end 408 and thereby separate the lead from fibrous scar tissue. Through such catheters the lead may be separated along its length, as well as separated at its distal end from fibrous scar tissue, thereby permitting the lead to be readily extracted from the body. In the herein described embodiment, additional fibers 412 may be employed as described above to allow the visualization of the location and the rapid positioning of the catheter in relation to the fibrous scar tissue or bone that may be holding the lead body in a fixed location.

The present invention provides a system and method for utilizing an infrared imaging system for placing leads or any other device within the coronary sinus, branch cardiac veins and/or specific locations in the heart. Although several specific embodiments are discussed herein for exemplary purposes, it will be understood other types of catheters may be utilized. For example, systems and methods such as disclosed in U.S. Pat. Nos. 6,122,552 and 5,639,276 incorporated herein by reference may usefully employ the current invention. The current invention may additionally be incorporated into a guide wire or a lead itself.

The preceding specific embodiments are illustrative of the practice of the invention. It is to be understood, therefore, that other expedients known to those of skill in the art or disclosed herein may be employed. In the following claims, means-plus-function clauses are intended to cover the structures described herein as performing the recited function and not only structural equivalents but also equivalent structures. For example, although a nail and a screw may not be structural equivalents in that a nail employs a cylindrical surface to secure wooden parts together, whereas a screw employs a helical surface, in the environment of fastening wooden parts, a nail and a screw are equivalent structures.

It is therefore to be understood that, within the scope of the appended claims, the invention may be practiced otherwise than as specifically described without actually departing from the spirit and scope of the present invention.

What is claimed is:

1. A system for guiding an implantable medical device into a cardiac vein or coronary artery of a body, comprising:
an elongated shaft adapted to be positioned within the cardiac vein or coronary artery;
a fiber optic cable suitable for transmitting light, the cable being proximate to at least a distal portion of the elongated shaft;
an infrared light source to transfer infrared light down the cable;
an optical head assembly coupled to the cable to transmit to and receive from the body the infrared light;
a sensing device to sense the infrared light received from the body via the optical head assembly; and
a device coupled to the sensing device to generate from the received infrared light an image indicative of a position of at least the distal portion of the elongated shaft when the elongated shaft is positioned within the body.

2. The system of claim 1 wherein said elongated shaft includes a guide wire and lead or a guide catheter and lead.

3. The system of claim 1 wherein said light is visible light.

4. The system of claim 1 wherein said light includes infrared light.

5. The system of claim 1 wherein said sensing device includes a camera and video signal processing system.

6. A lead navigation, delivery and location system in tortuous vasculatures incorporated with a vision system to display the lead and the vascular environment thereof, the system comprising:
a guide wire and lead;
visible light or infrared light detection means incorporated in the vision system; and
a sensor at a distal end of said lead for transmission of visual images to a receiver in the vision system.

7. The system of claim 6 wherein the lead is operably integrated with a guide catheter.

8. The system of claim 6 wherein said sensor includes an APS sensor.

9. The system of claim 6 wherein said transmission from said sensor includes optical fibers.

10. The system of claim 9 wherein said optical fibers are of size to allow higher wavelength.

11. The system of claim 6 wherein a laser lead extraction system is incorporated with the vision system.

12. The system of claim 6 wherein an ablation system is incorporated with the vision system.

13. A method for guiding an implantable medical device (IMD) into a cardiac vein or coronary artery of a body, comprising:
positioning an elongated shaft within the body, the elongated shaft including a fiber optic cable suitable for transmitting light;
transmitting infrared light down the cable and into the body;
receiving reflected infrared light from the body via an optical head assembly positioned at a distal end of the elongated shaft;
generating an image indicative of a position of the distal end of the elongated shaft from the reflected infrared light; and
using the image to guide the IMD into a cardiac vein or coronary artery.

14. The method of claim 13 wherein the IMD is placed in a pulmonary vein ostia.

15. The method of claim 14 wherein the location of the IMD is verified via one of measuring and stimulating.
